(19) United States
(12) Patent Application Publication
(10) Pub. No.: US 2013/0323323 A1
(43) Pub. Date: Dec. 5, 2013

(54) Therapeutic Method and Associated Compound for Augmenting the Liver's Metabolization of Oxygen-Modified Toxins

(71) Applicants: David T. Colina, Columbus, OH (US); Daniel J. Kim, Columbus, OH (US)

(72) Inventors: David T. Colina, Columbus, OH (US); Daniel J. Kim, Columbus, OH (US)

(21) Appl. No.: 13/907,672

(22) Filed: May 31, 2013

Related U.S. Application Data
(60) Provisional application No. 61/792,040, filed on Mar. 15, 2013.
(60) Provisional application No. 61/653,468, filed on May 31, 2012.

(51) Int. Cl. A61K 33/00; A23L 2/52; A61K 45/06

(52) U.S. Cl. CPC A61K 33/00; A61K 45/06; A23L 2/52
USPC 424/600

(57) ABSTRACT

A therapeutic method and associated composition for counteracting the negative and toxic physiological effects of the consumption or production of oxygen-modified toxins, such as alcohol, congeners, and associated metabolites, by augmenting or restoring the liver's ability to metabolize said elements, thereby preventing or abating the negative effects of the toxins, such as the symptoms of a hangover in the case of alcohol. Oxygen-infused water with a high solubilization level combined with caffeine, sodium, potassium, dextrose, or flavoring elements provides a therapeutic and pleasant dose before, during, or after the intake of alcohol, thereby increasing the metabolism of oxygen-modified toxin, alcohol, congener, and associated metabolites, replenishing nutrient levels deteriorated by toxin-introduction or alcohol consumption, and treating hangover symptoms.

### CROSS-REFERENCE TO RELATED APPLICATIONS

This application claims the benefit of U.S. provisional patent application No. 61/792,040 filed Mar. 15, 2013, and U.S. provisional patent application No. 61/653,468 filed May 31, 2012, the disclosures of which are both hereby incorporated by reference.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### INCORPORATION-BY-REFERENCE OF MATERIAL SUBMITTED ON A COMPACT DISC

Not applicable.

### BACKGROUND OF THE INVENTION

1. Field of the Invention

The present disclosure pertains to methods and formulations for generally reducing the presence and increasing the metabolic rate (or the rate at which toxins are metabolized in the body) of toxins in the body, and specifically for reducing or alleviating the symptoms that follow acute alcohol intoxication or other such oxygen-modified toxins and toxic by-products, by augmenting the liver's ability to metabolize toxins, alcohol and associated congeners, and generally oxygen-modified toxins, and further by treating some of the negative side effects that follow acute alcohol intoxication.

2. Description of Related Art Including Information Disclosed Under 37 C.F.R. 1.97 and 1.98

Ethanol (CH₃CH₃OH) is the type of alcohol found in alcohol beverages. It is a small, polar, water-soluble compound that is rapidly absorbed into the bloodstream via the gastrointestinal tract. Absorption of ethanol occurs mainly in the stomach and the distal small intestine. The rate of alcohol absorption is heavily influenced by the concentration and type of alcoholic beverage that is ingested, the rate at which it is ingested, the presence or absence of additional food in the gastrointestinal system, the composition of said food, and genetic and environmental variables.

Ethanol is a psychoactive chemical and one of the oldest known recreational drugs. Congeners, also known as fusel oils, are chemical substances such as acetone, esters, and aldehydes that are produced and concentrated during the fermentation of alcohol. They provide unique flavors, aromas, and colors to alcoholic beverages but also augment their intoxicating effects and contribute heavily to the symptoms of a hangover. Harmful metabolic derangements can be caused by excessive alcohol consumption, which include electrolyte imbalances, multiple vitamin deficiencies (A, D, E, folate, thiamine, riboflavin, B2, B6, B12), hypoglycemia, and lactic acidosis. Excessive alcohol consumption can also cause adverse cardiovascular abnormalities, which include peripheral and cerebral vasodilation, arrhythmias, hypovolemia, hypothermia, and hypotension. Central nervous system slowing, psychosis, inhibition of central respiratory drive, seizures, and direct nerve damage are all manifestations of alcohol's negative effects on the central nervous system. The gastrointestinal system may be negatively affected as well in the form of acute gastritis, gastric ulcers, alcohol-induced pancreatitis, alcoholic hepatitis, and cirrhosis.

The sequelae that follow acute alcohol intoxication—commonly referred to as a “hangover”—can include headache, nausea/vomiting, abdominal pain, fatigue, myalgia, hypersensitivity to sound, photophobia, dizziness, lightheadedness, fever/chills, diarrhea, decreased attention and awareness, irritability, insomnia, and dry mouth. These symptoms are a direct manifestation of the metabolic, cardiovascular, neurologic, and gastrointestinal derangements caused by alcohol and its associated congeners.

The physiologic elimination of alcohol and its associated congeners occurs via several pathways including gastric alcohol dehydrogenase (ADH), hepatic alcohol dehydrogenase, the microsomal ethanol oxidizing system (MEOS), catalase, and acetaldehyde dehydrogenase. These processes occur primarily in the liver, which like all other organs in the human body, relies on a process called aerobic respiration to generate energy. A continuous oxygen supply is essential for aerobic respiration to take place. Oxygen is therefore an essential component in the breakdown of alcohol and its associated congeners. Several studies have documented an increase in oxygen uptake in the livers of rats after exposure to ethanol as well as a significant decrease in the rate of ethanol elimination in environments that are low in oxygen. Moreover, increased rates of alcohol metabolism have been associated with increased oxygen delivery to the liver.

Alcohol is primarily metabolized by an enzyme called alcohol dehydrogenase, which oxidizes ethanol into a highly toxic metabolite called acetaldehyde. Acetaldehyde is then in turn broken down into acetic acid by an enzyme called aldehyde dehydrogenase, which also requires oxygen. In fact, the process by which most toxins (e.g., congeners) are broken down requires oxygen directly or indirectly. The oxidation of ethanol into acetate utilizes up to 60-80% of the total oxygen consumed by the liver. FIG. 1 illustrates these typical metabolic pathways used by the body when processing alcohol and its associated congeners and toxic byproducts.

Acetaldehyde is thought to be a major contributing factor in the development of hangovers due to its highly toxic effects on the body. In fact, a widely available pharmaceutical called Disulfiram, which is used by patients with alcohol dependence to help maintain sobriety, blocks the action of aldehyde dehydrogenase and causes an intentional and immediate toxic reaction if alcohol is consumed akin to a condensed hangover. This reaction occurs because of a rapid accumulation of acetaldehyde in the bloodstream. The FDA provides: “Disulfiram plus alcohol, even small amounts, produces flushing, throbbing in the head and neck, throbbing headache, respiratory difficulty, nausea, copious vomiting, sweating, thirst, chest pain, palpitation, dyspnea, hyperventilation, tachycardia, hypotension, syncope, marked uneasiness, weakness, vertigo, blurred vision, and confusion. In severe reactions there may be respiratory depression, cardiovascular collapse, arrhythmias, myocardial infarction, acute congestive heart failure, unconsciousness, convulsions, and death.” Not surprisingly, the largest concentration of aldehyde dehydrogenase is found in the stomach and the liver.

There is a unique relationship that exists between the liver's ability to metabolize alcohol and its ability to receive oxygen. The liver is the largest internal organ and gland in the human body. Its primary function is to metabolize and/or remove various substances found within circulating blood. Approximately 25% of the body's total blood volume flows through the liver every minute. A continuous blood supply is essential to every organ in the human body because blood delivers vital oxygen to living tissues in exchange for carbon dioxide.

The liver is unique in that it has a dual blood supply. The systemic circulation, which provides the liver with arterial blood, originates from the heart. Venous blood, which is high in carbon dioxide and low in oxygen, is pumped into the lungs where oxygen is absorbed and CO2 is released. Systemic blood is then pumped out into peripheral tissues and organs via arteries. Arterial blood is nearly 100% saturated with oxygen. This is why despite only providing the liver with 25% of its total blood supply, the systemic circulation is responsible for 50% of its total oxygen supply. The liver's second source of blood is called the portal venous circulation. It provides the liver with venous blood that originates from the gastrointestinal tract (stomach, intestines, and pancreas).

The makeup of portal venous blood is largely dependent on the contents of the gastrointestinal tract. Both nutrients and toxins are absorbed after ingestion and transported directly to the liver for processing before being distributed systemically. A large blood vessel called the portal vein serves as a collecting duct for blood that is delivered to the liver for filtering. Venous blood contains high levels of CO2 and low levels of O2 in comparison to arterial blood. This is why despite providing the liver with nearly three times the amount of blood that the systemic circulation supplies (75% of the total blood supply), the portal venous circulation only provides the liver with 50% of its total oxygen supply. Thus, blood supplied to the liver through the portal vein is relatively low in oxygen saturation (˜66% oxygen saturation compared to ˜100% oxygen saturation of systemic blood). FIG. 2 shows a simplified diagram illustrating the body's oxygen flow to and from the liver as described, with low-oxygen venous flow depicted by solid arrows and high-oxygen arterial flow depicted by outline arrows.

Inhalation of pure oxygen in an individual with normal lung function will have a minimal effect on the oxygen content of arterial blood because arterial blood is already nearly 100% saturated. Inhalation of pure oxygen will also have no effect on the oxygen content of portal venous blood because portal venous blood is not distributed to the lungs until after it flows through the liver and is subsequently returned to the heart. The oxygen content of portal venous blood can, however, be increased through the ingestion of solubilized oxygen in the form of an oxygen infused liquid. Additionally, multiple studies have documented a significant increase in the rate of ethanol elimination in both monkeys and humans after intravenously administered or ingested alcohol is followed by ingestion of oxygen infused fluids; increases in the rate of ethanol elimination as high as 60% have been reported. No measurable increase in the elimination rate of ethanol has been shown in studies where pure oxygen was administered to rats, cats, and dogs via inhalation.

One 2010 study conducted by a group of Korean researchers found that by infusing an alcohol beverage with oxygen, the alcohol in the beverage could be metabolized more quickly. Analysis of the data collected from the study showed that, generally, the ingestion of dissolved oxygen can increase the rate of alcohol metabolism. Specifically, the tabulated results showed that:
Time to descend to 0.050% blood alcohol level (BAL) after ingestion of a 360 cc alcohol beverage at 20 ppm oxygen was 34.8 min faster than after ingestion of the same volume of alcoholic beverage at 8 ppm oxygen, representing 23.8% faster recovery time.
Time to descend to 0.050% BAL after ingestion of a 360 cc alcohol beverage at 25 ppm oxygen was 31.1 min faster than after ingestion of the same volume of alcoholic beverage at 8 ppm oxygen, representing 19.2% faster recovery time.
Time to reach 0.000% BAL after ingestion of a 240 cc alcoholic beverage at 20 ppm oxygen was 20 minutes faster than after ingestion of the same volume of alcoholic beverage at 8 ppm oxygen, representing 7.8% faster recovery time.
Time to descend to 0.000% BAL for a 360 cc alcoholic beverage at 20 ppm oxygen was 23 minutes faster than after ingestion of the same volume of alcoholic beverage at 8 ppm oxygen, representing 6.6% faster recovery time.
Time to descend to 0.000% BAL for a 360 cc alcoholic beverage with 25 ppm was 27.1 minutes faster than after ingestion of the same volume of alcoholic beverage at 8 ppm oxygen, representing 7.3% faster recovery time.

These results suggest that, for the processes involved in metabolizing alcohol, increasing the amount of oxygen supplied to the liver will decrease the processing time of alcohol and its congeners. Therefore, the previously mentioned metabolic processes transpiring in the liver that are involved in the breakdown of both alcohol and its toxic byproducts could be enhanced by increasing the amount of oxygen available to the liver during alcohol consumption and its subsequent processing.

Several products that exist today claim to treat the symptoms arising from acute alcohol intoxication and commonly known as a “hangover.” Of these products, only one provides a theoretical pathway in which the metabolism of alcohol is augmented. All other products in this category only address some of the symptoms that are experienced during a hangover or replenish nutrients that may have been consumed during the metabolism of alcohol, but do not aim to increase the rate at which the liver metabolizes the toxic substances (i.e., alcohol, acetaldehyde, and associated congeners) that actually cause hangovers. More than just having significant morbidity, hangovers also have enormous societal and economic implications. Several studies spanning multiple countries including the United States, Canada, and The United Kingdom have estimated the cost of hangovers due to lost productivity and lost wages as high as $148 billion per year per country. Therefore, there is a significant existing need in the field for an effective product for, and method of, preventing, reducing, or alleviating the negative effects of alcohol consumption.

Alka Seltzer is an example of a commonly used compound that is meant to address some of the symptoms of a hangover. It consists of an effervescent tablet that contains 325 mg of acetylsalicyclic acid, 1000 mg citric acid, and 1916 mg of sodium bicarbonate and is meant to be ingested after being dissolved in water. The administration of this compound, however, operates only to reduce negative physiological reactions to alcohol processing, and fails to address their root cause.

A similarly formulated product called Blow Fish contains 500 mg acetylsalicyclic acid, sodium bicarbonate, and 60 mg caffeine. The acetylsalicyclic acid (aspirin) in these formulations blocks the production of a pro-inflammatory chemical called prostaglandin and is intended to treat headache and myalgia that may be experienced during a hangover. Sodium bicarbonate is intended to neutralize stomach acids and relieve gastrointestinal upset during a hangover. Caffeine (in Blow Fish) is both a central nervous stimulant and can provide headache relief through the action of central vasoconstriction.

Alka Seltzer and Blow Fish aim to provide symptomatic relief, but do nothing to increase the rate of elimination of the toxic substances that cause hangovers. There is in fact evidence that aspirin actually significantly lowers the body's ability to break down alcohol by interfering with the action of alcohol dehydrogenase. Furthermore, chronic use of non-steroidal anti-inflammatory drugs (NSAIDs) like aspirin inhibits the production of agents that protect the stomach's lining from acids. Aspirin also inhibits platelet aggregation, which makes it more difficult for blood to clot and bleeding to be controlled. Chronic NSAID use is therefore a well-known risk factor for peptic ulcer disease and gastrointestinal bleeding, which can be further aggravated by consumption of alcohol. Therefore, a need for an effective product for and method of reducing or alleviating the negative effects of alcohol consumption without introducing further health problems exists in the art.

Mercy is an example of a product that claims to prevent hangovers. It provides nutrients and antioxidants that may help to protect the liver during the process of alcohol metabolism without actually augmenting its ability to eliminate alcohol, alcohol's toxic metabolites, and associated congeners. Mercy comes in the form of a 250 ml beverage to be consumed during alcohol ingestion (one full can for every three to five cocktails according to manufacturer literature), and no more than three cans of Mercy are recommended per day. Mercy's active ingredients include potassium, sugar, magnesium, L-carnitine, milk thistle seed extract, N-acetylcysteine, an assortment of B vitamins, folic acid, vitamin C and chamomile extract.

Potassium and sugar are useful for their hydrating effects and in correcting hypoglycemia. Magnesium is an essential cofactor in the activation of hundreds of catalytic enzymes. L-carnitine can act as an antioxidant that protects against lipid perodixation and oxidative stress. Milk thistle seed extract has been shown to protect the liver against toxin-induced damage. N-acetylcysteine has also been shown to have protective effects on the liver, but may actually have the opposite effect if taken after the ingestion of alcohol. Vitamin B1—also known as thiamine—is essential to nerve transmission. Alcohol impairs the uptake of dietary thiamine in the gastrointestinal tract and can lead to a deficiency. Vitamin B3—also known as niacin—is an enzymatic cofactor that is depleted during the metabolism of alcohol. B12 plays a key role in maintaining normal nervous system function, and folic acid is important in the repair of DNA. Both can be easily depleted in those that consume alcohol frequently. Vitamin C has many functions including acting as an antioxidant and an immune system modulator. Chamomile extract has been reported to have anti-inflammatory properties and protective effects on the gastrointestinal tract.

Other products claim to provide hangover relief through the elimination of alcohol and its metabolites from the bloodstream through the use of alcohol absorbing materials. For example, Chaser, a product disclosed in U.S. Pat. No. 6,827,932 to R. Crippen et al., consists of activated charcoal and limestone powder encapsulated in gelatin capsules. Chaser claims to “prevent 17 hangover symptoms.” The active ingredients in Chaser are intended to absorb alcohol and congeners before they are absorbed, and are meant to be taken during the consumption of alcohol. Two pills are stated to last for three hours or up to six drinks. Once alcohol and congeners are absorbed however, Chaser is of no utility, and as previously noted, alcohol is rapidly absorbed into the gastrointestinal system. Chaser offers no mechanism by which the elimination of alcohol and congeners that have already been absorbed can be enhanced.

Still others claim to reduce hangover symptoms by boosting the detoxification of alcohol via a Phase II glucuronidation pathway in the body, such as U.S. Pat. No. 7,662,863 to Andrews et al. The glucuronidation pathway is one of at least six Phase II pathways that may be active during alcohol detoxification. The specific agent disclosed in Andrews, does not enhance the total alcohol detoxification process, and is therefore of limited use in the search for the cure for hangovers. There is a need in the art for a compound that provides a means for the enhanced processing of alcohol and its associated congeners, and that reduces or alleviates physiological effects of the process.

U.S. Pat. No. 5,747,079 to Hoffman discloses the use of oxygenated solutions to alleviate, reduce, control, or eliminate halitosis, but does not disclose the process for oxygenating such solutions or their use related to the alleviation of a hangover. The method of oxygenation comprised in said patent is by way of passing a stream of pressurized oxygen into a solution within a container until the desired level of dissolved oxygen concentration is achieved, after which the container is sealed under a pressure of 2.0 to 6.0 atmospheres. The container is usually made of glass or plastic. The disclosed range of oxygen concentration in the solution is 20-1000 mg/l, ideally 40-400 mg/l. No support exists in the Hoffman disclosure for achieving said ranges, however, nor are they believed to be physically achievable. There is a need, therefore, for a compound, method of manufacturing said compound, and associated therapeutic method for the treatment of the negative effects of acute alcohol intoxication that is not disclosed in the prior art.

Existing compositions and methods combat the negative effects of alcohol consumption by providing limited increases to the detoxification process, preventing the absorption of alcohol and congeners, protecting bodily systems and organs involved during their processing, or by treating the systemic physiological side effects of their processing. Thus, there is a need in the art for a compound for and method of eliminating alcohol and congeners from the body more rapidly, while simultaneously reducing those negative physiological effects and indirect societal costs associated with their natural elimination.

### BRIEF SUMMARY OF THE INVENTION

Exemplary embodiments of the present invention relate generally to oxygen-infused beverages for augmenting the metabolism of toxins and toxic byproducts within the body, wherein those toxins of a type modifiable and metabolized through the use of oxygen.

An aspect of the invention is a method for augmenting the metabolism of oxygen-modified toxins and their toxic byproducts comprising the steps of providing an oxygen-infused beverage to a human recipient for oral administration, said oral administration to occur immediately before, after, or during the toxin introduction event. The oxygen-infused beverage contains at least 30 ppm oxygen content, and preferably 60 ppm.

An aspect of the invention is a method for augmenting the metabolism of alcohol and its toxic byproducts comprising the steps of providing an oxygen-infused beverage to a human recipient for oral administration, said oral administration to occur immediately before, after, or during the alcohol consumption event. The oxygen-infused beverage contains at least 30 ppm oxygen content, and preferably 60 ppm.

An additional object of the invention includes the oxygen-infused beverage further containing at least one agent for reducing at least one physiological effect of a toxin introduction event selected from the group of sodium benzoate, potassium sorbate, sodium chloride, potassium chloride, monosodium phosphate, aspartame, and caffeine.

Yet another object of the invention is to provide a therapeutic composition for augmenting the metabolism of alcohol and its toxic byproducts comprised of at least 250 ml of water having at least 30 ppm solubilized oxygen content, at least one agent for reducing at least one physiological effect of an alcohol consumption event selected from the group of sodium benzoate, potassium sorbate, sodium chloride, potassium chloride, monosodium phosphate, aspartame, and caffeine, and one or more flavoring agents, wherein the at least 250 ml of water having at least 30 ppm solubilized oxygen content, the at least one agent for reducing the at least one physiological effect of an alcohol consumption event, and one or more flavoring agents are combined under pressure into a container comprising glass or aluminum.

Yet another object of the invention is to provide a therapeutic composition for augmenting the metabolism of oxygen-modified toxins and their toxic byproducts comprised of at least 250 ml of water having at least 30 ppm solubilized oxygen content, at least one agent for reducing at least one physiological effect of a toxic introduction event selected from the group of sodium benzoate, potassium sorbate, sodium chloride, potassium chloride, monosodium phosphate, aspartame, and caffeine, and one or more flavoring agents, wherein the at least 250 ml of water having at least 30 ppm solubilized oxygen content, the at least one agent for reducing the at least one physiological effect of a toxin introduction event, and one or more flavoring agents are combined under pressure into a container comprising glass or aluminum.

The composition can further comprise an oxygen carrier compound, wherein the oxygen carrier compound promotes oxygen binding thereby increasing the solubilized oxygen content.

Another aspect of the invention is a method for making an ingestible therapeutic compound for augmenting metabolism of alcohol, its byproducts, and associated congeners, and for reducing at least one physiological effect of an alcohol consumption event, comprising the steps of, attaching a chilled, filtered, and pressurized water source to a liquid inlet on a carbonator having a gas inlet and pressurized outlet hose, attaching a pressurized oxygen source to the gas inlet on the carbonator wherein the operation of the carbonator will produce oxygenated water at the pressurized outlet hose, combining at least one agent for reducing the at least one physiological effect of an alcohol consumption event with at least one flavoring agent by dissolving them in water to form an additive syrup, acidifying the additive syrup, carbonating the additive syrup with carbon dioxide, cooling the additive syrup after carbonation by passing it through a chilled syrup outlet hose, attaching the pressurized outlet hose of the carbonator and the chilled syrup outlet hose to a dispenser, combining the oxygenated water and the additive syrup with the dispenser to form the compound, dispensing the compound into a glass or aluminum bottle, and sealing the glass or aluminum bottle under a pressure greater than two atmospheres. A portable apparatus practicing this method is also disclosed.

In addition to the novel features and advantages mentioned above, other benefits will be readily apparent from the following descriptions of the drawings and exemplary embodiments.

### BRIEF DESCRIPTION OF THE SEVERAL VIEWS OF THE DRAWINGS

Novel features and advantages of the present invention, in addition to those mentioned above, will become apparent to those skilled in the art from a reading of the following detailed description in conjunction with the accompanying drawings wherein identical reference characters refer to identical parts and in which:

FIG. 1 is a diagram of the relevant circulatory elements of the human body;

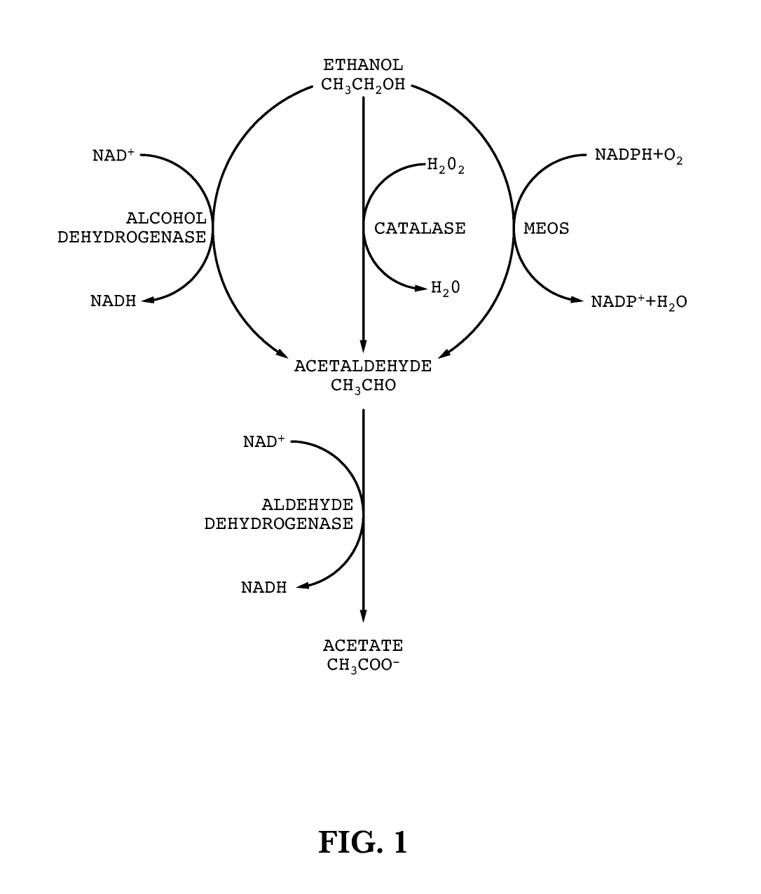

FIG. 2 depicts typical processes involved in the metabolism of alcohol and its toxic byproducts; and

FIG. 3 is a flow diagram of the steps taken to create the disclosed therapeutic compound.

### DETAILED DESCRIPTION OF THE INVENTION

Alcohol and its toxic byproducts are broken down in the cells of the liver by several enzymatic pathways including those mediated by alcohol dehydrogenase, the microsomal ethanol oxidizing system (MEOS), catalase, and acetaldehyde dehydrogenase. The liver and every other organ in the body rely on a process called cellular respiration to function properly. In cellular respiration, biochemical energy from nutrients (carbohydrates, amino acids, fatty acids) is converted into ATP and waste products such as CO2. Cellular respiration encompasses a series of catabolic reactions that involve the oxidation of said nutrients into different chemical substrates. Cellular respiration in the cells of the human liver occurs via aerobic respiration. Oxygen serves as a final electron acceptor in aerobic respiration. The liver therefore requires a continuous supply of oxygen to function properly.

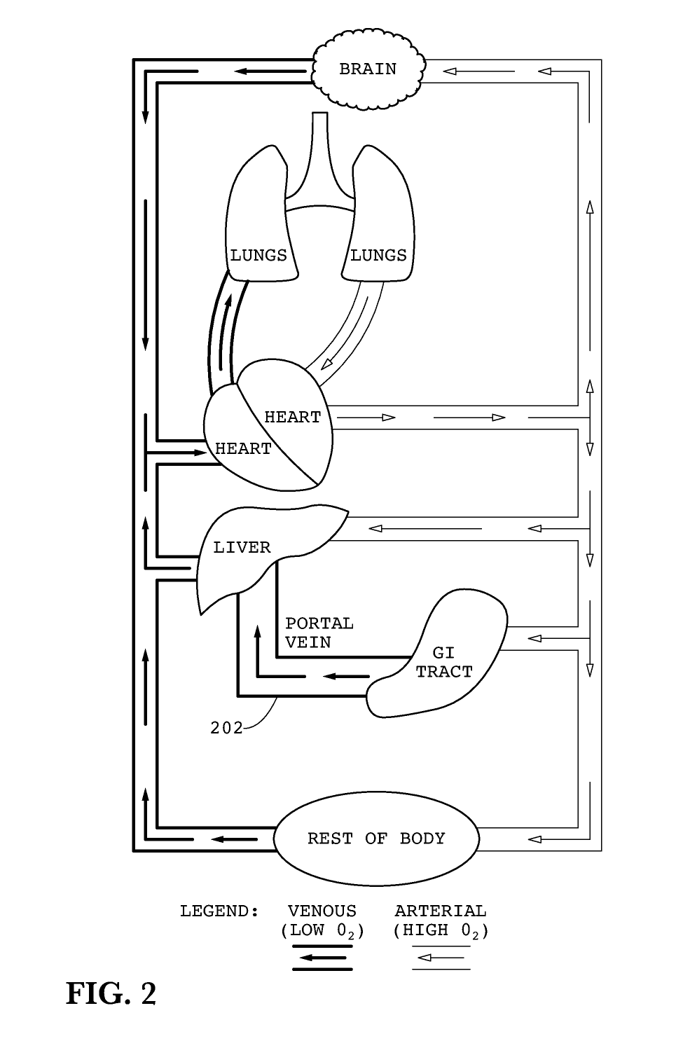

During and after excessive alcohol consumption, the liver's energy requirement increases as it is presented with a larger than normal level of alcohol and toxins to metabolize. In order to meet the liver's increased energy demands, its oxygen supply must also rise accordingly. The oxygen supply to the liver is limited, however, in that only 25% of the blood that it receives is fully saturated with oxygen (100 mmHg) from the lungs and systemic circulation. The other 75% comes by way of the portal venous system, which contains blood that is substantially lower in oxygen content (40 mmHg). With such a limited supply of oxygen, the rate at which alcohol is metabolized reaches a plateau (typically around 0.02 g alcohol/100 ml blood per hour). The present disclosure teaches augmentation of the liver's metabolizing ability by increasing the oxygen content delivered to the liver via the portal vein pathway 202, shown in FIG. 2.

Normally, the body increases its heart rate and respiratory rate during periods when it requires more energy, e.g., during exercise. However, alcohol blunts the body's ability to respond to the liver's increased energy demand by acting as a central nervous system depressant. As a result, the heart rate and respiratory rate are often significantly decreased, which further limits the liver's oxygen supply. The next most useful route in which additional oxygen can be supplied to the liver is via ingestion. The current disclosed compounds and associated methods provide solubilized oxygen to the liver via absorption into portal venous blood by the GI tract. An increase in the amount of oxygen delivered to the liver augments or restores its ability to metabolize alcohol and its toxic byproducts, thereby mitigating the adverse physiologic effects caused by excessive alcohol consumption.

Generally, the present disclosure refers to the metabolism of alcohol and its associated congeners as a specific target for enhanced metabolic processes. However, the liver systematically metabolizes a variety of toxins, which are produced endogenously via the body's natural metabolic processes or originate exogenously and enter the body through processes such as but not limited to ingestion inhalation, intravenous injection, intramuscular injection, rectal absorption, and dermal absorption. There are two primary phases involved in detoxification. Phase I involves modification of toxic substances through oxidation, reduction, or hydrolysis. Phase II involves conjugation of toxic substances. A large and diverse group of phase I enzymes collectively called the cytochrome P450 (CYP) system is responsible for metabolizing the majority of toxins. CYP enzymes use oxygen to modify toxic compounds, drugs, and steroid hormones. The MEOS enzymatic pathway, for example, is a part of the CYP family of enzymes, and takes on a larger role in metabolizing alcohol-related toxins in chronic, heavy drinkers. The present disclosure is meant to apply to methods and products for increasing the metabolism of both endogenously and exogenously produced oxygen-modified toxins. The term “oxygen-modified toxin” is meant to encompass endogenous and exogenous toxins for which the body requires significant utilization of oxygen to metabolize.

To provide a useful compound through which oxygen can be delivered to the liver, the invented compounds and methods are embodied in an oxygen-infused beverage that is capable of being ingested. The compound contains at least 30 ppm oxygen content which when ingested orally will be absorbed in the gastrointestinal system and delivered via the portal venous system, thereby significantly increasing the body's ability to process and metabolize alcohol, its associated congeners, and the byproducts resulting from detoxification. The compound thus reduces the toxic effects of excess alcohol consumption while increasing the elimination of harmful byproducts from the body, thereby reducing hangover symptoms associated with excess alcohol consumption. Applicant's compound is preferably bottled at a level of oxygen content of at least 60 ppm, well over double the oxygen levels found in the Korean study and over 8 times the amount of oxygen found in untreated water.

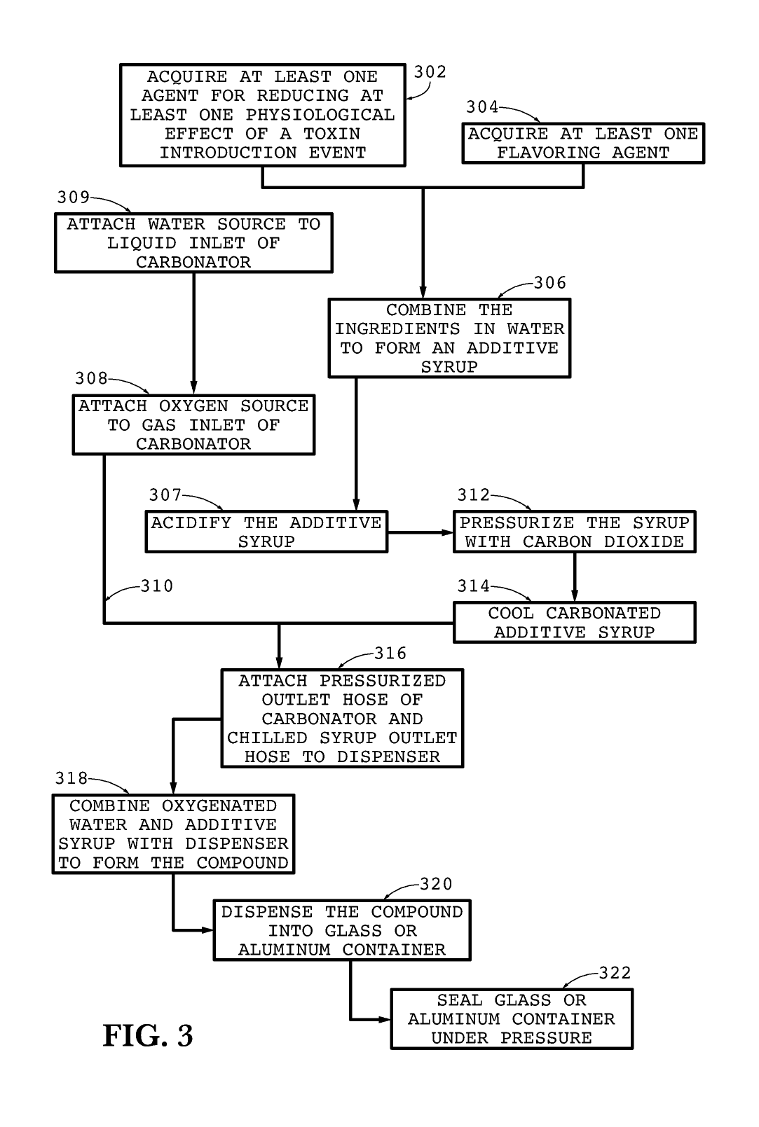

To make the disclosed compound, a medical grade or aviation grade pressurized oxygen source is used and connected to the gas inlet of a carbonator apparatus, as at block 309 in FIG. 3. A cooled, filtered water source is connected to the liquid inlet of the carbonator, as at block 309. The water source may be cooled by, for example, passing the water through a chill plate, a coil of hose placed in ice, or any similar combination that brings the water passed to the carbonator to a temperature near 0 to 8 degrees Celsius. Oxygenation of the water occurs when the carbonator is turned on and oxygen is forced under pressure into the water that is present with the pressurized tank after a period of at least 15 minutes.

The carbonator pressurized outlet house carries the mixture away from the carbonator for later processing as disclosed herein, via arrow 310. Alternatively, large pressurized containers of water can be infused with oxygen and stored for later processing, allowing the mixture to reach an equilibrium with the highest possible oxygen content given the environmental conditions present.

The best mode of the invention makes use of a McCann's “Big Mac” carbonator (model #E400397), although many other commercial embodiments may be used, or any other similar process derived for this purpose. For example, the use of the McCann carbonator utilizes oxygen pressurized to about 2,200 psi and regulated to between 100 psi to 120 psi during the oxygen-infusion stage. The filtered water source is pressurized between 50 psi and 100 psi. However, various pressures and temperature combinations can be utilized, as those skilled in the art will recognize, depending on the equipment used to increase the oxygen content of water above naturally occurring levels. It is essential that the water in this process be contained in a highly pressurized environment when combined with the oxygen, as this is a key to increasing the solubility of oxygen within the water.

The solubility of oxygen in water is not only dependent on pressure as noted above, but also on temperature. The normal solubility of oxygen in water at 25 degrees Celsius and 1 standard atmosphere is 6 ppm. At 5 degrees Celsius and 1 standard atmosphere, the solubility of oxygen in water increases approximately 50% to 9 ppm. Therefore, production and storage of the disclosed compound should be carried out at low temperatures and at high pressures.

A mixture of flavoring agents (e.g., extracts) are combined to form an additive syrup, as at blocks 302, 304, and 306 shown in FIG. 3. The additive syrup is also acidified, as at block 307. The use of a Flojet syrup pump to pressurize with carbon dioxide, as at block 312, and deliver the additive syrup to a dispenser, via arrow 310, is considered the best mode at the time of disclosure, however any comparable method to pressurize the additive syrup is considered encompassed by the disclosed methods. The additive syrup is then cooled to approximately match the temperature of the oxygenated water, as at block 314. The cooling of the additive syrup can occur before or after pumping, as needed. The additive syrup and oxygenated water are then combined in a dispenser, as at block 318, by connecting the outlet of the syrup pump and the carbonator pressurized outlet hose to the dispenser, as at block 316. The best mode of the invention utilizes a Wunder Bar bar gun calibrated to yield a 5:1 ratio of oxygenated water to syrup.

The invention is dispensed, as at block 320 directly into a 500 ml container (e.g., bottle or can) that is generally oxygen impermeable using the dispenser. Care is taken to leave a minimal amount of headspace at the top of the container before it is sealed, as at block 322, with an adhesive-backed pressure sensitive foam liner that is contained within a plastic cap so that oxygen loss to the atmosphere is reduced. Glass or coated aluminum are currently considered best mode materials in the bottling of the invention due to their low cost and ability to prevent diffusion of dissolved oxygen out of the container, and to prevent leaching of potentially harmful chemicals from plastic containers into the invention. Glass and aluminum are superior to plastic in retaining oxygen in a solution due to the fact that they are significantly less porous. The overall areas allowing diffusion of plastic and glass have been estimated to be 6×10−2−3×10−1 cm2/cm2 and 3×10−7−8×10−5 cm2/cm2, respectively. This means that solubilized oxygen is as much as 150 times more likely to diffuse across plastic compared when compared to glass. Both DEHP and DEHA are potentially harmful to multiple organ systems in the human body including the central nervous system and the endocrine system and have been detected in the urine of test subjects who have consumed products contained in different types of plastic bottles. Metal containers, such as conventional aluminum cans or containers similar to the aluminum bottle developed by the Daiwa company, which have internal and external lamination films and provide superior gas barrier properties over plastic containers are also suitable for bottling of the invention.

The compositions disclosed herein may also be further enhanced by the addition of oxygen carrier compounds. Complex molecules such as the heme group reversibly bind oxygen. The addition of complex or simple molecules to the composition could be utilized to further enhance the oxygen delivery efficacy of the composition. Certain oxygen binding compounds are also known to be pH sensitive. Such pH sensitive compounds are known in the art, and could be chosen based on their ability to bind oxygen in the bottled composition, but to release the bound oxygen in the environment of the gut or stomach.

The present disclosed compounds and methods may also operate to synergistically alleviate the negative effects of alcohol consumption by not only providing an increased metabolic ability to process alcohol, its congeners, and associated toxic byproducts, but also by providing for the therapeutic alleviation of the physiological side effects of alcohol consumption. For example, dehydration is a major factor in the symptoms that occur following excessive alcohol consumption. It occurs due to the inhibitory effect of ethanol on an important chemical messenger in the body called antidiuretic hormone (ADH). When secreted by the brain, ADH signals the kidneys to conserve water. This occurs naturally when the body senses that it is in a low volume state. Without ADH signaling to the kidneys, the brain cannot limit urine production in lieu of conservation of water. The only way to reduce the inhibitory effect of ethanol on ADH is to eliminate ethanol from the system. The invented compositions and methods disclosed herein not only promote the elimination of alcohol from the body, but also replenish it with electrolyte-rich water. The recommended serving size of the disclosed compounds is approximately 500 ml, a standard volume for intravenously administered fluid boluses during fluid resuscitation of a hypovolemic/dehydrated patient in a hospital setting. More or less of the compound can be administered during the consumption of alcohol depending upon the needs of the subject, their genetic makeup, the amount of alcohol consumed, and how quickly and the length of time during which the alcohol is consumed. The addition of agents to counteract such negative physiological effects also applies to the physiological effects arising from oxygen-modified toxin introduction events generally.

Electrolytes, most notably sodium and potassium, are essential for normal cellular function, which in large part consists of osmotic homeostasis. Osmotic homeostasis is achieved by ensuring that the correct proportions of sodium and potassium are maintained inside and outside of a cell. Nearly all cellular membranes contain sodium/potassium pumps that are constantly working to achieve this balance. Plasma in a healthy human body contains a specific proportion of sodium and potassium and is therefore osmotically balanced to be the ideal environment for cells to live in.

Normal saline is a synthetic fluid designed to be osmotically similar to plasma. Intravenous infusion of normal saline along with dextrose and potassium is a fundamental principle in rapid fluid resuscitation of a hypovolemic/dehydrated patient. The electrolyte content of the invention (16% DV sodium, 8% DV potassium) is based off the gold standard IV maintenance fluids in the hospital setting (D5 0.45 NS+20 K). The disclosed compound and methods therefore not only augment the elimination of alcohol via ingested oxygen, but enable the body to rehydrate by the replenishment of electrolytes via ingested sodium and potassium.

Cerebral vasodilation and central nervous system (CNS) slowing caused by alcohol and its toxic byproducts also contribute heavily to the symptoms of a hangover. Cerebral vasodilation can cause headaches and lightheadedness. Peripheral vasodilation can cause body temperature dysregulation. CNS slowing can cause fatigue, dizziness, and decreased attention and awareness. Caffeine is a naturally occurring substance that is nearly 100% bioavailable and therefore rapid-acting with a peak plasma concentration within 30-45 minutes of ingestion. Caffeine has many useful effects on the cardiovascular and central nervous systems, which include intrinsic analgesic properties, central vasoconstriction, and CNS excitability. Caffeine promotes wakefulness through the antagonism of adenosine receptors in the brain, which normally inhibit the release of excitatory neurotransmitters. Caffeine is also a potent central vasoconstrictor and can therefore provide immediate relief from headaches. Caffeine can also augment stimulation to pleasure centers in the brain by preventing the breakdown of dopamine. The addition of caffeine, therefore, can be used to provide many therapeutic benefits alongside of the augmented liver processing ability, further increasing the efficacy of the compounds in treating hangover symptoms.

Dextrose is a simple sugar that is used by cells as a primary source of energy. It is an essential component in cellular respiration. The benefits of dextrose in a resuscitative solution (in the right quantity) are three fold. Firstly, dextrose may increase the absorption of fluids and electrolytes. Glucose and sodium are both absorbed by the same membrane channel in the lining of the proximal small intestine. Sodium uptake is therefore promoted by glucose uptake via activation of a common transporter. Increased sodium absorption in turn creates an osmotic gradient that facilitates fluid absorption. Several studies support this concept and have shown that the hydrating ability of fluids that contain up to 10% dextrose in addition to electrolytes are superior to or at least equal to those of fluids that contain electrolytes alone. In intravenous applications, dextrose is added to IV fluids in order to maintain isotonicity without increasing sodium or potassium content.

The second benefit of dextrose lies in its role as a source of fuel. Excessive alcohol consumption and binge drinking often take place over extended periods of time and can disrupt the timing of meals or alter ones appetite. After extended periods of activity without adequate nourishment, the body's carbohydrate stores can be depleted. Alcohol can also induce hypoglycemia, especially in diabetics or those that are malnourished or have compromised liver function, by diverting pancreatic blood flow and causing increased production of insulin. Dextrose can both replenish carbohydrate stores and combat hypoglycemia.

Lastly, dextrose is a common sweetener used in the food industry. Its addition to a resuscitative solution can aid in creating a flavor profile that is more palatable and therefore easier to ingest. Dextrose can therefore be used as a compound element to increase the flavor appeal of the compound, provide energy, and increase rehydration processes.

These and other such additive elements can be combined with the oxygenated portion of the compound to further increase the efficacy of the compound in treating hangover symptoms. The efficacy can be increased by adding elements known in the art to reduce hangover symptoms such as headaches, nausea, central nervous system slowing, fatigue, myalgia, and other such metabolic, cardiovascular, neurologic, and gastrointestinal derangements. The addition of these elements will complement the alleviation of hangover symptoms caused by the liver's augmented ability to process and metabolize alcohol, its associated congeners, and toxic byproducts. Dry powdered ingredients embodying these traditional hangover remedies can be dissolved into water along with the flavoring agents to create the additive syrup, thereby enhancing the efficacy of the compound.

Additive elements in addition to those listed herein that are known in the art or used as traditional therapeutic elements for reducing negative physiological effects related to a toxin introduction event may be utilized in the creation of an oxygenated compound as described herein. Other additive elements, now known or later developed or used, that enhance or augment positive physiological functions may also be combined with an oxygenated based to enhance oxygen-modified toxin metabolism and increase normal bodily functions. For example, a wide array of nutritional supplements, including but not limited to, proteins, vitamins, minerals, fiber, hormones, and fatty and amino acids, may be utilized as enhancer agents within the compound.

The disclosed compound can be used whenever an alcohol consumption event occurs, i.e., when a subject has or may drink alcohol excessively. It may also be used to increase the metabolic rate of other generally oxygen-modified toxins when a toxin introduction event occurs, i.e., during spikes in concentration, such as for recovery from physical activity or processes during which oxygen-modified toxins are products, or after ingesting oxygen-modified toxins other than alcohol. It should be administered by oral consumption one or more times, as needed, during a period temporally close to the alcohol consumption event (or a similar toxin consumption or production event, i.e., a toxin introduction event), such that the increase in oxygen delivered to the liver will have a metabolic effect on the alcohol processing time. Therefore, the compound can be administered before, in conjunction with, or after the consumption of alcohol.

A preferred embodiment of the disclosed therapeutic compound includes oxygen levels of greater than 60 ppm. The embodiment also contains the desired additive agents of sodium (14-16% recommended daily value), potassium (7-8% DV), and caffeine (100-160 mg). Sweeteners may optionally include, for example, dextrose, erythritol, cane sugar, stevia, or monk fruit.

Any embodiment of the present invention may include any of the optional or preferred features of the other embodiments of the present invention. The exemplary embodiments herein disclosed are not intended to be exhaustive or to unnecessarily limit the scope of the invention. The exemplary embodiments were chosen and described in order to explain some of the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention. It is the intention, therefore, to limit the invention only as indicated by the scope of the claims.

What is claimed is:

1. A method for augmenting the metabolism of at least one oxygen-modified toxin comprising the steps of providing an oxygen-infused beverage to a human recipient for oral administration, said oral administration to occur immediately before, after, or during a toxin introduction event.

2. The method of claim 1 wherein the at least one oxygen-modified toxin is alcohol and its toxic byproducts, and the toxin introduction event is an alcohol consumption event.

3. The method of claim 1 wherein the oxygen-infused beverage contains at least 30 ppm oxygen content.

4. The method of claim 2 wherein the oxygen-infused beverage contains at least 30 ppm oxygen content.

5. The method of claim 3 wherein the oxygen-infused beverage further contains at least one agent for reducing at least one physiological effect of the toxin introduction event selected from the group of sodium benzoate, potassium sorbate, sodium chloride, potassium chloride, monosodium phosphate, aspartame, and caffeine.

6. The method of claim 4 wherein the oxygen-infused beverage further contains at least one agent for reducing at least one physiological effect of the alcohol consumption event selected from the group of sodium benzoate, potassium sorbate, sodium chloride, potassium chloride, monosodium phosphate, aspartame, and caffeine.

7. A therapeutic composition for augmenting the metabolism of at least one oxygen-modified toxin comprised of:
at least 250 ml of water having at least 30 ppm solubilized oxygen content;
at least one agent for reducing at least one physiological effect of a toxin introduction event selected from the group of sodium benzoate, potassium sorbate, sodium chloride, potassium chloride, monosodium phosphate, aspartame, and caffeine; and
one or more flavoring agents,
wherein the at least 250 ml of water having at least 30 ppm solubilized oxygen content, the at least one agent for reducing the at least one physiological effect of the toxin introduction event, and one or more flavoring agents are combined under pressure into a container comprising glass or aluminum.

8. The therapeutic composition of claim 7 wherein the at least one oxygen-modified toxin is alcohol and its toxic byproducts, and the toxin introduction event is an alcohol consumption event.

9. The composition of claim 7 further comprising an oxygen carrier compound, wherein the oxygen carrier compound promotes oxygen binding thereby increasing the solubilized oxygen content.

10. The composition of claim 8 further comprising an oxygen carrier compound, wherein the oxygen carrier compound promotes oxygen binding thereby increasing the solubilized oxygen content.
